(19) United States
(12) Patent Application Publication
(10) Pub. No.: US 2015/0105658 A1
(43) Pub. Date: Apr. 16, 2015

(54) ULTRASONIC IMAGING APPARATUS AND CONTROL METHOD THEREOF

(71) Applicant: SAMSUNG ELECTRONICS CO., LTD., Suwon-si (KR)

(72) Inventors: Sung Chan PARK, Suwon-si (KR); Joo Young KANG, Yongin-si (KR); Kyu Hong KIM, Seoul (KR); Jung Ho KIM, Yongin-si (KR); Su Hyun PARK, Hwaseong-si (KR)

(73) Assignee: SAMSUNG ELECTRONICS CO., LTD., Suwon-si (KR)

(21) Appl. No.: 14/515,629

(22) Filed: Oct. 16, 2014

(30) Foreign Application Priority Data
Oct. 16, 2013 (KR) 10-2013-0123558

(51) Int. Cl. A61M 37/00; A61K 49/22; A61B 8/00; A61B 8/08

(52) U.S. Cl. CPC A61M 37/0092; A61B 8/481; A61K 49/223; A61B 8/5223; A61B 8/085; A61B 8/461
USPC 600/431

(57) ABSTRACT

Disclosed herein are an ultrasonic imaging apparatus for enhancing target therapy, and a control method thereof. The ultrasonic imaging apparatus includes: an inputter configured to receive a command for setting, in a first ultrasound image, a target area of an object to which target therapy is to be applied, the first ultrasound image showing a lesion to which ultrasound contrast agents including therapeutic agents have been bound within the object; and an image processor configured to compare the first ultrasound image to a second ultrasound image acquired after ultrasonic waves have been irradiated to a target part corresponding to the target area, and to detect at least one of an area into which the therapeutic agents have been delivered and an amount of the therapeutic agents delivered into the area.

### CROSS-REFERENCE TO RELATED APPLICATION

This application claims the benefit of Korean Patent Application No. 10-2013-0123558, filed on Oct. 16, 2013 in the Korean Intellectual Property Office, the disclosure of which is incorporated herein by reference in its entirety.

### BACKGROUND

1. Field

Exemplary embodiments of the present disclosure relate to an ultrasonic imaging apparatus, and a control method thereof, and more specifically, to an ultrasonic imaging apparatus for enhancing target therapy, and a control method thereof.

2. Description of the Related Art

Medical imaging apparatuses include an X-ray imaging apparatus, a fluoroscopy system, a Computerized Tomography (CT) scanner, a Magnetic Resonance Image (MRI) apparatus, Positron Emission Tomography (PET), and an ultrasonic imaging apparatus.

The ultrasonic imaging apparatus irradiates ultrasonic waves to the inside of an object, and receives ultrasonic echoes reflected from the inside of the object so as to non-invasively acquire section images about the inner tissues of the object or images about blood vessels of the object based on the ultrasonic echo.

The ultrasonic imaging apparatus has advantages that it is a compact, low-priced apparatus compared to other medical imaging apparatuses and it can display images in real time. Also, the ultrasonic imaging apparatus has safety benefits since there is no risk for patients to be exposed to radiation such as X-rays. For the advantages, the ultrasonic imaging apparatus is widely used to diagnose the heart, breasts, abdomen, urinary organs, uterus, etc.

### SUMMARY

Therefore, it is an aspect of the exemplary embodiments to provide an ultrasonic imaging apparatus for enhancing target therapy, and a control method thereof.

Additional aspects of the exemplary embodiments will be set forth in part in the description which follows and, in part, will be obvious from the description, or may be learned by practice of the exemplary embodiments.

In accordance with an aspect of an exemplary embodiment, there is provided an ultrasonic imaging apparatus including: an inputter configured to receive a command for setting, in a first ultrasound image, a target area of an object to which target therapy is to be applied, the first ultrasound image showing a lesion to which ultrasound contrast agents including therapeutic agents have been bound within the object; and an image processor configured to compare the first ultrasound image to a second ultrasound image acquired after ultrasonic waves have been irradiated to a target part corresponding to the target area, and to detect at least one of an area into which the therapeutic agents have been delivered and an amount of the therapeutic agents delivered into the area.

In accordance with another aspect of an exemplary embodiment, there is provided an ultrasonic imaging apparatus including: a display configured to display a first ultrasound image showing a lesion to which ultrasound contrast agents including therapeutic agents have been bound within an object, and a registered image obtained by registering a predetermined target image with the first ultrasound image; an inputter configured to receive a command for setting, in the first ultrasound image, a target area to which target therapy is to be applied to the object; and an image processor configured to compare the first ultrasound image to a second ultrasound image acquired after ultrasonic waves have been irradiated to a target part corresponding to the target area, and to detect at least one of an area into which the therapeutic agents of the ultrasound contrast agents have been delivered, and an amount of the therapeutic agents delivered into the area.

Therefore, by comparing an ultrasound image acquired before target therapy to an ultrasound image acquired after target therapy to detect and display at least one of an area into which therapeutic agents have been put and an amount of the therapeutic agents put into the area, an operator can accurately check an amount of therapeutic agents put into lesion tissue.

Since an amount of therapeutic agents delivered into lesion tissue can be accurately checked, accuracy of target therapy can be improved.

### BRIEF DESCRIPTION OF THE DRAWINGS

These and/or other aspects of the exemplary embodiments will become apparent and more readily appreciated from the following description of the exemplary embodiments, taken in conjunction with the accompanying drawings of which:

FIG. 1 illustrates a cross section of a particle constructing an ultrasound contrast agent;

FIG. 2 is a view for describing a concept of target therapy using ultrasound contrast agents;

FIG. 3 is a perspective view of an ultrasonic imaging apparatus according to an exemplary embodiment;

FIG. 4 is a block diagram of an ultrasonic imaging apparatus according to an exemplary embodiment;

FIG. 5 illustrates a configuration of a transmit beamformer of an ultrasonic imaging apparatus;

FIG. 6 illustrates a configuration of a receive beamformer of an ultrasonic imaging apparatus;

FIG. 7 is a block diagram of an image processor of an ultrasonic imaging apparatus, according to an exemplary embodiment;

FIGS. 8, 9, and 10 show examples of images output as the results of image processing by an image processor during target therapy; and

FIG. 11 is a flowchart illustrating a control method of an ultrasonic imaging apparatus, according to an exemplary embodiment.

### DETAILED DESCRIPTION

Reference will now be made in detail to the exemplary embodiments, examples of which are illustrated in the accompanying drawings, wherein like reference numerals refer to like elements throughout.

Hereinafter, exemplary embodiments of an ultrasonic imaging apparatus and a control method thereof will be described with reference to the accompanying drawings.

The ultrasonic imaging apparatus irradiates ultrasonic waves to a target area of an object, and converts ultrasonic echoes reflected from the target area of the object into electrical signals. Then, the ultrasonic imaging apparatus acquires an ultrasound image about the target area based on the electrical signals.

Ultrasound contrast agents may be used for ultrasonic diagnosis using the ultrasonic imaging apparatus. The ultrasound contrast agents are used to clearly represent a lesion, for example, tumor tissue in an ultrasound image. The ultrasound contrast agents may be put into a vein of an object before ultrasonic diagnosis.

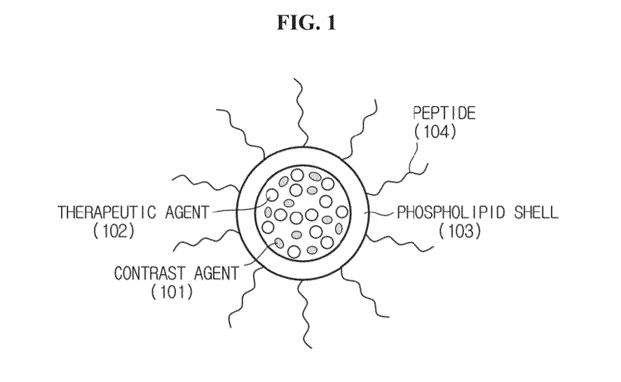

FIG. 1 illustrates a cross section of a particle constructing an ultrasound contrast agent. Generally, a particle of an ultrasound contrast agent is constructed with contrast agents 101, therapeutic agents 102, and a phospholipid shell 103 encapsulating the contrast agents 101 and the therapeutic agents 102. Peptides 104 and antibodies are bound to the outer surface of the phospholipid shell. The peptides and antibodies can be bound to tumor tissue.

The ultrasound contrast agent can be classified into a microparticle contrast agent and a nanoparticle contrast agent according to its particle size.

An example of the microparticle contrast agent is a microbubble. Examples of the nanoparticle contrast agent include a perfluorocarbon (PFC) nanodroplet, a polyactic acid (PLA) nanobubble, a solid nanoparticle, and a liposome.

The microbubble has a size of 1 μm to 4 μm. Generally, the microbubble includes a phospholipid shell such as PFC, trapping gas.

The PFC nanodroplet has a size of 200 nm to 400 nm. The shell of the PFC nanodroplet may be made of PLA.

The PLA nanobubble has a size of 40 nm to 200 nm, and the solid nanoparticle has a size of 20 nm to 100 nm. The solid nanoparticle can be detected by ultrasonic waves because of small amounts of gas trapped in cavities.

The liposome has a size of 20 nm to 1 μm. The liposome is constructed with an amphiphilic bilayer surrounding an aqueous core.

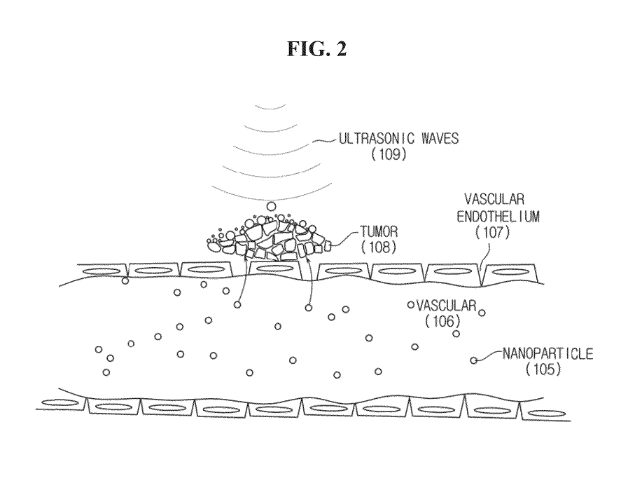

The ultrasound contrast agents may be used for target therapy, as well as for increasing the contrast of an ultrasound image. Target therapy using ultrasound contrast agents will be described in more detail with reference to FIG. 2, below.

FIG. 2 is a view for describing a concept of target therapy using ultrasound contrast agents.

Nanoparticles 105, such as a PFC nanodroplet, a PLA nanobubble, a solid nanoparticle, and a liposome, can leak out of the vascular endothelium 106 and enter the extravascular space since the nanoparticles 105 have small sizes. Nanoparticles existing in the extravascular space 106 may coalesce into collections with sizes of microns. Particles with sizes of microns may be bound to tumor tissue 108, and detected by ultrasonic waves 109. As a result, tumor tissue 108 can be clearly represented in an ultrasound image. Accordingly, an operator can check the tumor tissue in the ultrasound image, and select an area in which the tumor tissue 108 is located, as a target area to which target therapy will be applied. Then, the operator may operate an ultrasonic imaging apparatus to irradiate ultrasonic waves 109 to a target part corresponding to the selected target area. If the ultrasonic waves 109 are irradiated to the target part, nanoparticles 105 in the target part burst by the ultrasonic waves 109, so that therapeutic agents encapsulated by the nanoparticle shells are delivered to the tumor tissue 108.

Meanwhile, although not illustrated in FIG. 2, microbubbles cannot leak out of the vascular endothelium and are trapped in the intravascular space since the microbubbles have larger sizes than nanoparticles. That is, the microbubbles are trapped in the intravascular space around the tumor tissue. Such microbubbles may be detected by ultrasonic waves. If microbubbles are found in an ultrasound image, an operator may select an area in which the microbubbles are found, as a target area. Thereafter, the operator may operate the ultrasonic imaging apparatus to irradiate ultrasonic waves to a target part corresponding to the selected target area. If ultrasonic waves are irradiated to the target part, nanoparticles in the target part are burst by the ultrasonic waves, so that therapeutic agents encapsulated by the nanoparticle shells leak out of the vascular endothelium and then are delivered to the tumor tissue.

A structure of an ultrasound contrast agent, and a concept of target therapy using the ultrasound contrast agents have been described above. Hereinafter, an ultrasonic imaging apparatus that can be used for target therapy will be described.

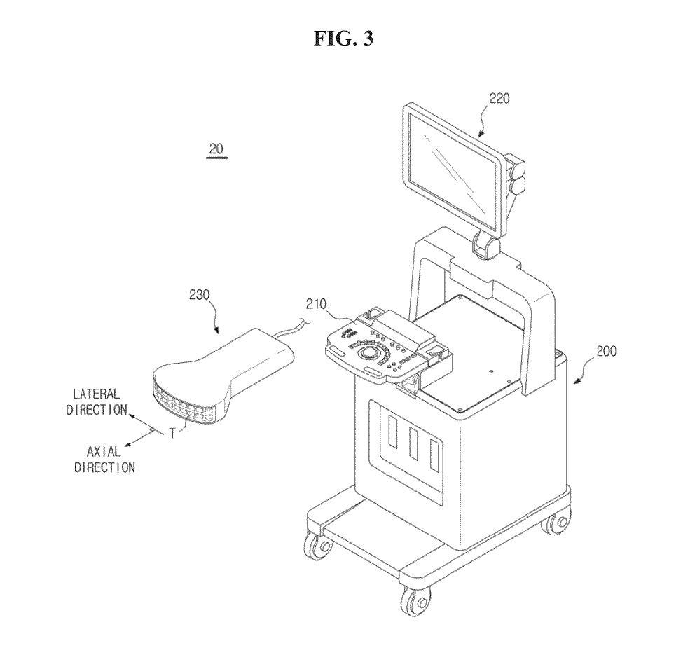

FIG. 3 is a perspective view of an ultrasonic imaging apparatus according to an exemplary embodiment.

As illustrated in FIG. 3, an ultrasonic imaging apparatus 20 may include a main body 200, an input unit 210 (e.g., inputter), a display unit 220 (e.g., display), and a probe 230.

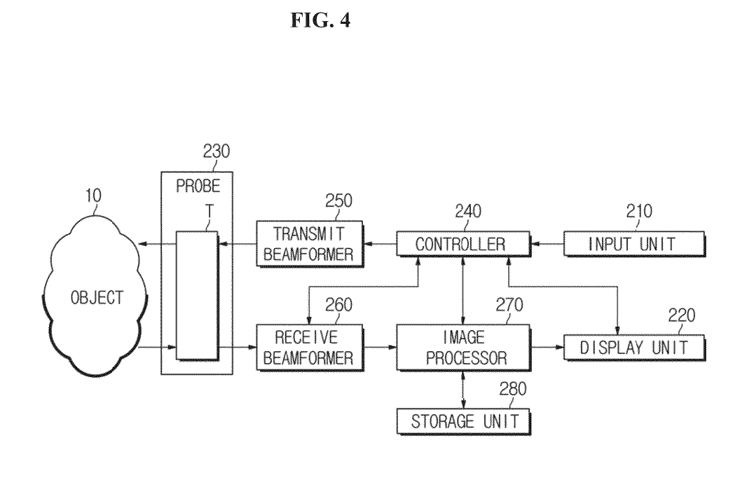

The main body 200 may accommodate main components of the ultrasonic imaging apparatus 20. For example, referring to FIG. 4, the main body 200 may accommodate a controller 240, a transmit beamformer 250, a receive beamformer 260, an image processor 270, and a storage unit 280 (e.g., storage). The individual components will be described in more detail with reference to FIG. 4, later.

The input unit 210 allows an operator to input an instruction or a command for manipulating the ultrasonic imaging apparatus 20. For example, the operator may input a diagnosis start command, a command for selecting an area to be diagnosed, a command for selecting a diagnosis type, and a command for selecting a type of an image to be displayed through the display unit 220, a command for selecting a target part to which target therapy is to be applied, a command for irradiating ultrasonic waves to the selected target part, and a command for selecting a mode for an ultrasound image, through the input unit 210.

The diagnosis type may include general diagnosis and diagnosis for target therapy. The general diagnosis may be an ultrasonic diagnosis which does not use ultrasound contrast agents, whereas the diagnosis for target therapy may be an ultrasonic diagnosis using ultrasound contrast agents for target therapy.

The type of the image to be displayed through the display unit 220 may include an ultrasound image acquired by the ultrasonic imaging apparatus 20, and a medical image acquired by a medical imaging apparatus having a different modality from the ultrasonic imaging apparatus 20. The medical imaging apparatus having the different modality from the ultrasonic imaging apparatus 20 may include a fluoroscopy system, a Computerized Tomography (CT) scanner, a Magnetic Resonance Image (MRI) apparatus, and Positron Emission Tomography (PET). In the following description, an image acquired by any one of these medical imaging apparatuses is referred to as a “non-ultrasound image”.

The mode for the ultrasound image may include an Amplitude mode (A-mode), a Brightness mode (B-mode), and a Motion mode (M-mode).

The input unit 210 allows an operator to input an instruction or a command for operating the ultrasonic imaging apparatus 20. The input unit 210 may include at least one of, for example, a keypad, a keyboard, a foot switch, and a foot pedal.

For example, the keyboard may be implemented as hardware, and mounted on the upper part of the main body 200. The keyboard may include at least one(s) of a switch(s), a key(s), a wheel, a joystick, a trackball, and a knop. The foot switch or the foot pedal may be disposed below the main body 200. The operator may control a part of functions of the ultrasonic imaging apparatus 200 using the foot pedal.

As another example, the keyboard may be implemented as software such as a Graphic User Interface (GUI). A keyboard implemented as software may be displayed through the display unit 220.

The display unit 220 may display at least one of an ultrasound image and a non-ultrasound image. The operator may set a type of an image to be displayed through the display unit 220, through the input unit 210. For example, the operator may set an image type such that only an ultrasound image is displayed through the display unit 220, or such that an ultrasound image and a non-ultrasound image corresponding to the ultrasound image are displayed at the same time through the display unit 220.

When the operator sets an image type such that all of an ultrasound image and a non-ultrasound image are displayed, the operator may set the ultrasound image to a main image, and the non-ultrasound image to a sub image. The main image and the sub image may be displayed by various methods. For example, the main image and the sub image may be arranged side by side in left and right directions in a display area of the display unit 220. As another example, the main image may be displayed in the entire display area, and the sub image may overlap a part of the main image. As another example, the main image may be displayed in the entire display area, and an icon for displaying the sub image may be displayed in the lower part of the display area. In this case, if the icon located in the lower part of the display area is selected, the sub image may be displayed in the entire display area, and an icon for displaying the main image may be displayed in the lower part of the display area.

The operator may manipulate the input unit 210 to change a setting for a main image and a sub image, or a setting for a display method for displaying a main image and a sub image, before or during ultrasonic diagnosis.

Meanwhile, there may be provided a plurality of display units. The display unit 220 may have only a display function or have both a display function and an input function. If the display unit 220 is a touch screen, the display unit 220 may have both a display function and an input function.

The probe 230 contacts an object 10 (see FIG. 4). One or more ultrasonic elements T are installed in one end of the probe 230. The ultrasonic elements T irradiate ultrasonic waves to the inside of the object 10, receive ultrasonic echo reflected from the inside of the object 10, and convert the ultrasonic echo into an electrical signal. For example, each ultrasonic element T may include an ultrasonic generator to generate ultrasonic waves and an ultrasonic reception device to receive ultrasonic echoes and convert the ultrasonic echoes into an electrical signal. As another example, the ultrasonic element T itself may generate ultrasonic waves and receive ultrasonic echoes.

The ultrasonic elements T may include ultrasonic transducers. A transducer is a device for converting a first type of energy into a second type of energy. For example, the ultrasonic transducer may convert electrical energy into wave energy, or wave energy into electrical energy. In particular, the ultrasonic transducers may perform all functions of an ultrasonic generator and an ultrasonic receiver.

In more detail, the ultrasonic transducers may include a piezoelectric material or a piezoelectric thin film. If alternating current power from an external power supply or from an internal power storage unit, for example, a battery, is applied to the piezoelectric material or the piezoelectric thin film, the piezoelectric material or the piezoelectric thin film vibrates at a specific frequency so that a specific frequency of ultrasonic waves are generated according to the vibration frequency. Meanwhile, if an ultrasonic echo having a specific frequency arrives at the piezoelectric material or the piezoelectric thin film, the piezoelectric material or the piezoelectric thin film vibrates according to the frequency of the ultrasonic echo. At this time, the piezoelectric material or the piezoelectric thin film outputs alternating current which corresponds to the vibration frequency.

Each ultrasonic transducer may be a magnetostrictive ultrasonic transducer which uses the magnetostrictive effect of a magnetic material, a piezoelectric ultrasonic transducer which uses the piezoelectric effect of a piezoelectric material, or a capacitive micromachined ultrasonic transducer (CMUT) that transmits and receives ultrasonic waves by using a vibration of several hundreds or thousands of micromachined thin films. However, the ultrasonic transducer may include any other type of ultrasonic transducer which is capable of generating ultrasonic waves according to electrical signals, or which is capable of generating electrical signals according to ultrasonic waves.

The ultrasonic transducers may be arranged in a linear array or in a convex array at the end part of the probe 230. In this case, the ultrasonic transducers may be arranged in a line or in a matrix form. If the ultrasonic transducers are arranged in a line, by moving the probe 230 in a scan direction, a plurality of ultrasound images may be acquired. If the ultrasonic transducers are arranged in a matrix form, by transmitting ultrasonic waves at once, a plurality of ultrasound images may be acquired.

Although not shown in the drawings, a cover for covering the ultrasonic transducers may be provided.

FIG. 4 is a block diagram of an ultrasonic imaging apparatus according to an exemplary embodiment.

Referring to FIG. 4, an ultrasonic imaging apparatus 20 may include the input unit 210, the display unit 220, the controller 240, the transmit beamformer 250, the probe 230, the receive beamformer 260, the image processor 270, and the storage unit 280. The controller 240, the transmit beamformer 250, the receive beamformer 260, the image processor 270, and the storage unit 280 may be accommodated in the main body 200 (see FIG. 3) of the ultrasonic imaging apparatus 20.

The input unit 210 and the probe 230 have been described above with reference to FIG. 3, and accordingly, further descriptions thereof will be omitted.

The controller 240 may control overall operations of the ultrasonic imaging apparatus 20. In detail, the controller 240 may generate a control signal for controlling at least one of the transmit beamformer 250, the receive beamformer 260, the image processor 270, the storage unit 280, and the display unit 220, according to an instruction or command received through the input unit 210. Also, the controller 240 may generate a control signal for controlling individual components according to an instruction or a command received from an external device through wired or wireless communication.

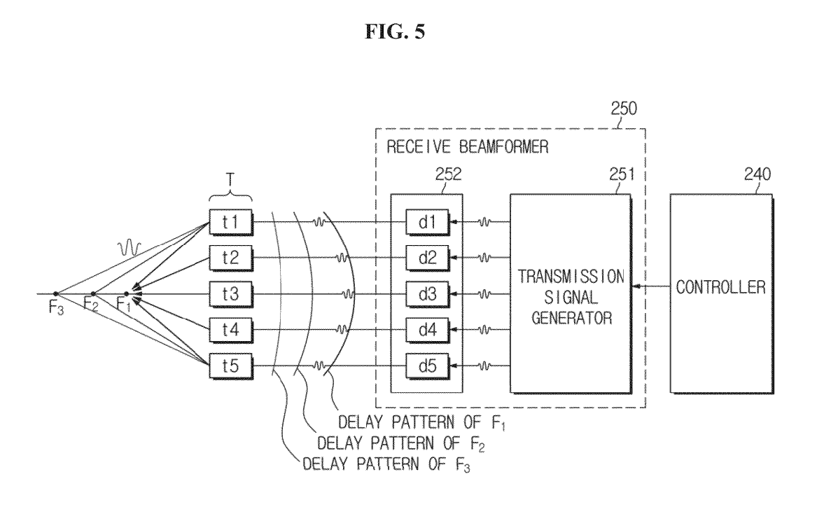

The transmit beamformer 250 may perform transmit beamforming. The transmit beamforming is performed in order to focus ultrasonic waves which are generated by one or more ultrasonic elements T to a focal point. That is, the transmit beamforming may be performed by coordinating the ultrasonic elements T to generate ultrasonic waves in an appropriate order in order to compensate for time differences with which ultrasonic waves generated by the ultrasonic elements T arrive at the focal point. The transmit beamforming will be described in more detail with reference to FIG. 5, below.

FIG. 5 illustrates a configuration of the transmit beamformer 250. As illustrated in FIG. 5, the transmit beamformer 250 may include a transmission signal generator 251 and a time delay unit 252 (e.g., time delayer).

The transmission signal generator 251 may generate transmission signals (such as, for example, high-frequency alternating current signals) that are to be applied to the ultrasonic elements T based on a control signal which is received from the controller 240. The transmission signals generated by the transmission signal generator 251 are provided to the time delay unit 252.

The time delay unit 252 delays each transmission signal generated by the transmission signal generator 251 in order to adjust a time at which the transmission signal arrives at the corresponding ultrasonic element T. If the transmission signals delayed by the time delay unit 252 are applied to the ultrasonic elements T, the ultrasonic elements T generate ultrasonic waves which correspond to the frequencies of the transmission signals. The ultrasonic waves generated by the ultrasonic elements T are focused onto a focal point. The location of the focal point at which the ultrasonic waves generated by the ultrasonic elements T are focused may vary based on the type of delay pattern that is applied to the transmission signals.

In more detail, in the exemplary embodiment of FIG. 5, five ultrasonic elements t1 to t5 are provided, and three delay patterns that can be applied to transmission signals are respectively represented by using thick solid lines, medium solid lines, and thin solid lines, respectively.

When the delay pattern represented as the thick solid lines is applied to transmission signals generated by the transmission signal generator 251, ultrasonic waves generated by the ultrasonic elements t1 to t5 are focused onto a first focal point F₁.

When the delay pattern represented as the medium solid lines is applied to transmission signals generated by the transmission signal generator 251, ultrasonic waves generated by the ultrasonic elements t1 to t5 are focused onto a second focal point F₂ which is more distant than the first focal point F₁.

When the delay pattern represented as the thin solid lines is applied to transmission signals generated by the transmission signal generator 251, ultrasonic waves generated by the ultrasonic elements t1 to t5 are focused onto a third focal point F₃ which is more distant than the second focal point F₂.

As described above, the location of a focal point varies based on the type of delay pattern that is applied to transmission signals generated by the transmission signal generator 251. Accordingly, when a delay pattern is applied, ultrasonic waves that are to be applied to an object are focused onto a fixed focal point (fixed-focusing). However, when two or more different delay patterns are applied, ultrasonic waves that are to be applied to an object are focused onto several focal points (multi-focusing).

As such, ultrasonic waves generated by the individual ultrasonic elements T are fixed-focused onto a single focal point, or multi-focused at several focal points. The focused ultrasonic waves are directed to the inside of an object. The ultrasonic waves directed to the inside of the object are reflected from a target area of the object. An ultrasonic echo which is reflected from the target area is received by the ultrasonic elements T. Then, the ultrasonic elements T convert the received ultrasonic echo into electrical signals. Hereinafter, the converted electrical signals will be simply referred to as reception signals (ultrasound echo signals). The reception signals output from the ultrasonic elements T are amplified and filtered, then converted into digital signals, and provided to the receive beamformer 260.

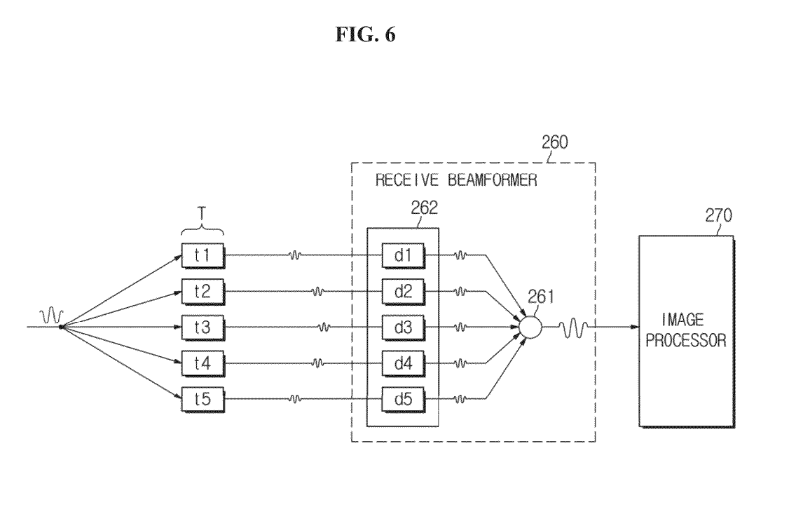

Referring again to FIG. 4, the receive beamformer 260 may perform receive beamforming on the reception signals which are converted into the digital signals. The receive beamforming is performed in order to correct time differences between reception signals output from individual ultrasonic elements T, and then to focus the resultant reception signals. The receive beamforming will be described in more detail with reference to FIG. 6, below.

FIG. 6 is a block diagram of the receive beamformer 260, according to an exemplary embodiment. Referring to FIG. 6, the receive beamformer 260 may include a time-difference corrector 262 and a focusing unit 261 (e.g., focuser).

The time-difference corrector 262 delays a respective reception signal which is output from each ultrasonic element T by a predetermined time period so that the reception signals output from the individual ultrasonic elements T can be transferred to the focusing unit 261 at the same time.

The focusing unit 261 may focus the reception signals based on the time-difference correction which is performed by the time-difference corrector 262. In particular, the focusing unit 261 may focus the reception signals after allocating a predetermined weight (such as, for example, a beamforming coefficient) to each reception signal in order to enhance or attenuate the corresponding reception signal with respect to the other reception signals. The focused reception signal may be provided to the image processor 270.

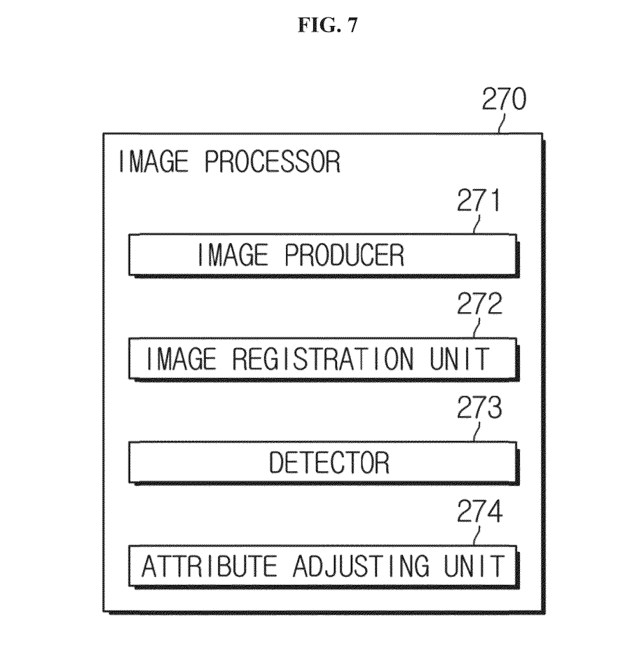

Referring again to FIG. 4, the image processor 270 may process ultrasound images in real time, and perform image registration. In addition, the image processor 270 may create a User Interface (UI) needed for target therapy. The image processor 270 will be described in more detail with reference to FIGS. 7 to 10, below.

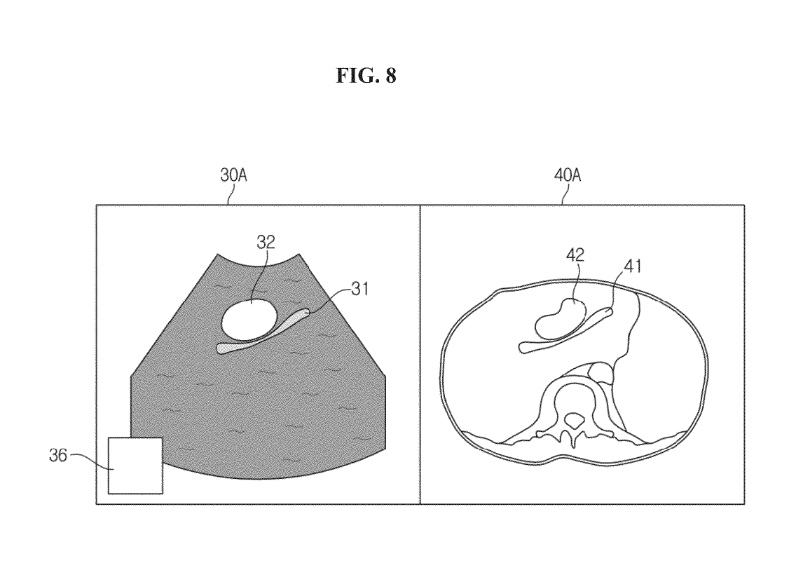

FIG. 7 is a block diagram of the image processor 270, according to an exemplary embodiment. FIGS. 8, 9, and 10 show examples of images output as the results of image processing by the image processor 270 during target therapy.

Referring to FIG. 7, the image processor 270 may include an image producer 271, an imaging registration unit 272, a detector 273, and an attribute adjusting unit 274 (e.g., attribute adjustor).

The image producer 271 may produce an ultrasound image based on the reception signals converted into digital signals. The ultrasound image produced by the image producer 271 may include one of more of a 2-Dimensional (2D) ultrasound image and a 3-Dimensional (3D) ultrasound image. The 2D ultrasound image may be a section image about the inside tissue of the object 10 (see FIG. 4). The 3D ultrasound image may be an image acquired by performing volume rendering on volume data generated based on a plurality of section images with respect to a specific viewpoint. The 2D and 3D ultrasound images may be black-and-white images and color images. A kind of an ultrasound image that is produced by the image producer 271 may vary depending on an instruction or a command input before or during ultrasonic diagnosis.

The image registration unit 272 may perform image registration. When the object 10 is photographed by different apparatuses, at different times, or at different viewpoints, images are obtained on different coordinate systems. The image registration is for deforming different images to represent the different images on a single coordinate system. By performing image registration, a matching relationship between images acquired by different methods can be understood.

Image registration may be classified into mono-modality registration and multi-modality registration. The mono-modality registration is to register images acquired by the same kind of apparatus. The multi-modality registration is to register images acquired by different kinds of apparatuses.

If the image registration unit 272 performs image registration with respect to an ultrasound image acquired by the ultrasonic imaging apparatus 20 and an ultrasound image acquired in advance, the image registration can be understood as mono-modality registration. If the image registration unit 272 performs image registration with respect to an ultrasound image acquired by the ultrasonic imaging apparatus 20 and a non-ultrasound image acquired in advance, the image registration can be understood as multi-modality registration.

In the following description, an image that is used as a reference image between two images to be subject to image registration is referred to as a “source image”. An image that is resampled for registration with a source image is referred to as a “target image”. A target image registered with a source image is referred to as a “registered image”.

According to an exemplary embodiment, a source image can be understood as an ultrasound image that is acquired in real time through the ultrasonic imaging apparatus 20. More specifically, a source image can be understood as an ultrasound image acquired before target therapy among ultrasound images that are acquired in real time through the ultrasonic imaging apparatus 20.

A target image may be an ultrasound or non-ultrasound image acquired in advance. The operator may manipulate the input unit 210 to select a target image before ultrasonic diagnosis. Also, the operator may manipulate the input unit 210 to change a type of a target image during ultrasonic diagnosis.

For example, an ultrasound image 30A and a registered image 40A (see FIG. 8) may be displayed side by side. As another example, the ultrasound image 30A and the registered image 40A may be displayed to overlap each other, after at least one attribute selected from at least one attribute of the ultrasound image 30A and at least one attribute of the registered image 40A is adjusted. Attributes of an image may include transparency and color.

The attribute adjusting unit 274 may adjust attributes of an image according to settings by the operator. The operator may set a display method of displaying two images, may indicate which image is subject to attribute adjustment when two images are displayed to overlap each other, and may set a degree to which an attribute is adjusted, through the input unit 210. The settings may be done before ultrasonic diagnosis. Also, setting values may be changed by the operator during ultrasonic diagnosis. In the following description, an example in which the ultrasound image 30A and the registered image 40A are displayed side by side will be described.

The detector 273 may compensate for ultrasonic attenuation of the ultrasound image 30A acquired before target therapy. The ultrasonic attenuation compensation may have occurred in at least one direction of an axial direction and a lateral direction.

Thereafter, the detector 273 may detect a lesion from the ultrasound image 30A subject to compensation for ultrasonic attenuation, and make the detected lesion overlap with the registered image 40A at the corresponding location in the registered image 40A. Since a lesion to which ultrasound contrast agents have been bound is represented brightly in an ultrasound image, the detector 273 may detect an area including at least one pixel having a brightness value greater than a reference value, or an area including at least one pixel having a brightness value belonging to a reference range, in the ultrasound image 30A, and determine the detected area as a lesion. Herein, the reference value or the reference range has been decided in advance through an experiment, and then stored in the storage unit 280 which will be described later.

The ultrasound image 30A, and the registered image 40A with which the lesion detected from the ultrasound image 30A overlaps may be displayed in a display area of the display unit 220. FIG. 8 shows a case in which the ultrasound image 30A, and the registered image 40A with which a lesion 32 detected from the ultrasound image 30A overlaps are displayed side by side in left and right directions in a display area.

In addition, the detector 273 may detect an amount of ultrasound contrast agents bound to the lesion. It is assumed that the sizes of particles constructing ultrasound contrast agents are uniform, and an amount of contrast agents and an amount of therapeutic agents existing in the shell of each particle are the same. In this case, the greater an amount of ultrasound contrast agents bound to a lesion, the brighter the lesion is shown in an ultrasound image. Accordingly, by analyzing the brightness of the lesion 32 detected from the ultrasound image 30A, an amount of ultrasound contrast agents bound to the lesion 32 can be detected.

Brightness of ultrasound contrast agents which are displayed in an ultrasound image according to an amount of ultrasound contrast agents may be decided in advance through an experiment, and may be stored in the form of a look-up table in the storage unit 280 which will be described later.

Meanwhile, ultrasonic waves attenuate in an axial direction and in a lateral direction. Accordingly, as tissue is located deeper from the skin of the body or more distant in the lateral direction, the tissue is shown darker in an ultrasound image. Accordingly, there is a need for compensating for ultrasonic attenuation before using the look-up table.

An example of a method of compensating for ultrasonic attenuation is to increase the brightness of tissue depending on depth through Time Gain Compensation (TGC). As another example, there is a method of tracing and detecting a single ultrasound contrast agent to record brightness when one ultrasound contrast agent exists at a predetermined location, and detecting an amount of ultrasound contrast agents based on the recorded brightness.

The detector 273 may detect an amount of ultrasound contrast agents bound to the lesion with reference to the look-up table, after compensating for ultrasonic attenuation with respect to the ultrasound image 30A. Information about the detected amount of ultrasound contrast agents may be displayed through an information display window 36. The information display window 36 may be displayed in a separate area from those of the ultrasound image 30A and the registered image 40A, or overlap with the ultrasound image 30A or the registered image 40A. FIG. 8 shows a case in which the image display window 36 overlaps with the ultrasound image 30A.

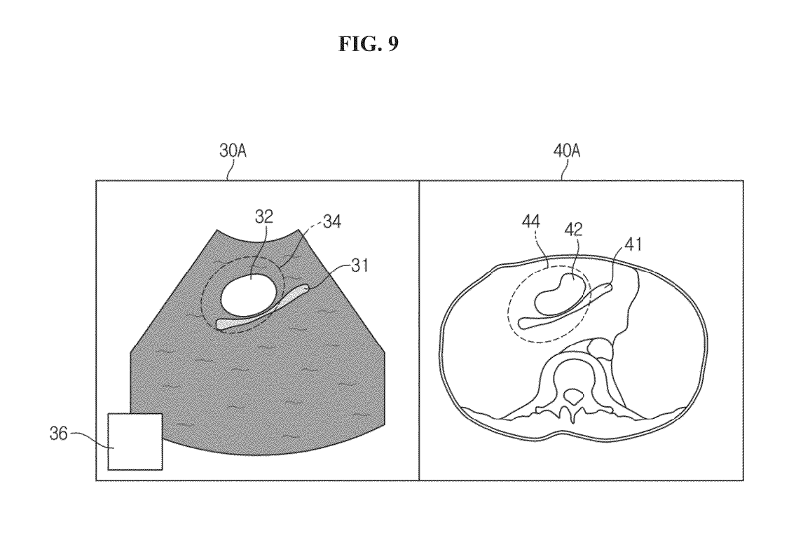

When the ultrasound image 30A and the registered image 40A are displayed side by side as shown in FIG. 8, the operator may set a target area 34 (see FIG. 9) that is applied to target therapy in the ultrasound area 30A. For example, the operator may drag and drop a cursor displayed in the display area using the input unit 210 (e.g., a mouse) to set the target area 34. If the display unit 220 is a touch screen, the operator may draw a desired range on the display area using his or her finger or a stylus pen to thus set the target area 34.

The target area 34 set in the ultrasound image 30A may be defined by an area dividing line. The area dividing line is an indicator for distinguishing the target area 34 from the remaining area in the ultrasound image 30A. For example, the area dividing line may be any one of a dotted line, a broken line, an alternate long and short dash line, an alternate long and tow short dashes line, and a solid line. Referring to FIG. 9, the target area 34 set in the ultrasound image 30A is defined by an area dividing line which is a dotted line.

Also, an area 44 of the registered image 40A, corresponding to the target area 34 of the ultrasound image 30A, may be defined by an area dividing line. Referring to FIG. 9, the area 44 of the registered image 40A, corresponding to the target area 34 of the ultrasound image 30A, is defined by an area dividing line which is a dotted line.

After the target area 34 is set in the ultrasound image 30A, target therapy of irradiating ultrasonic waves to a target part corresponding to the target area 34 may be performed. For example, the target therapy may be performed automatically when setting the target area 34 is completed. As another example, the target therapy may be performed automatically after a predetermined time period has elapsed from when setting the target area 34 has been completed. As still another example, the target therapy may be performed when a predetermined instruction or command is received through the input unit 210 after the target area 34 is set. Whether to perform target therapy automatically or manually, and a time interval until target therapy is performed after the target area 34 is selected, may be selected by the user.

If ultrasonic waves are irradiated to the target part, all or a part of ultrasound contrast agent particles bound to the lesion bursts, and therapeutic agents in the shells of the particles are delivered to the lesion. As such, since the ultrasound contrast agent particles burst by the ultrasonic waves irradiated to the target part, an ultrasound image acquired after the target therapy becomes different from the ultrasound image 30A acquired before the target therapy.

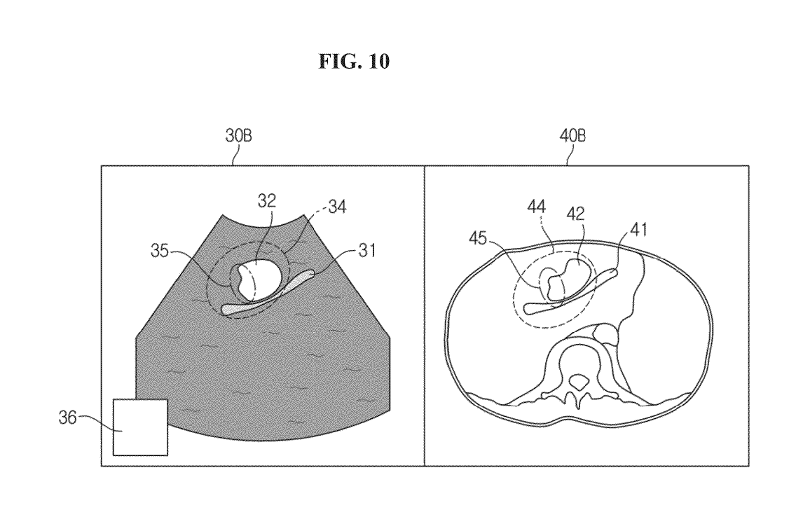

The detector 273 may compare the ultrasound image 30A acquired before the target therapy to an ultrasound image 30B (see FIG. 10) acquired after the target therapy, thereby detecting an area into which the therapeutic agents have been put, and an amount of the therapeutic agents which are put into the corresponding area.

In detail, the detector 273 may compensate for ultrasonic attenuation of the ultrasound image 30B acquired after the target therapy. Then, the detector 273 may compare the ultrasound images 30A and 30B to determine whether an area with reduced brightness is found in the ultrasound image 30B acquired after the target therapy. If an area with reduced brightness is found in the ultrasound image 30B, the detector 273 detects the area with the reduced brightness as an area 35 into which the therapeutic agents have been put. In addition, the detector 273 may determine a reduced degree in brightness of the area 35, and detect an amount of therapeutic agents put into the corresponding area 35 based on the result of the determination.

The area 35 into which the therapeutic agents have been put may be defined by an area dividing line in the ultrasound area 30B. For example, the area dividing line may be any one of a dotted line, a broken line, an alternate long and short dash line, an alternate long and tow short dashes line, and a solid line. For example, the area dividing line defining the area 35 into which the therapeutic agents have been put may have a different line type from the area dividing line defining the target area 34. As another example, the area dividing line defining the area 35 into which the therapeutic agents have been put may have the same line type as, and a different color from, the area dividing line defining the target area 34.

Also, an area 45 of the registered image 40B, corresponding to the area 35 of the ultrasound image 30B into which the therapeutic agents have been put, may be defined by an area dividing line. The areas 35 and 45 of the ultrasound image 30B and the registered image 40B, into which the therapeutic agents have been put, may be defined by the same type of area dividing lines. Referring to FIG. 10, the areas 35 and 45 of the ultrasound image 30B and the registered image 40B are defined by area dividing lines which are dotted lines. As such, by displaying area dividing lines corresponding to the areas 35 and 45 into which the therapeutic agents have been put, an operator can easily distinguish the areas 35 and 45 into which the therapeutic agents have been put.

Meanwhile, information about an amount of the therapeutic agents which are put into the detected area 35 may be displayed through the information display window 36. The information about the amount of the therapeutic agents which are put into the detected areas 35 may be displayed through the information display window 36, together with information acquired before target therapy, for example, information about a location of the lesion, and information about an amount of ultrasound contrast agents bound to the lesion. FIG. 10 shows a case in which the information display window 36 overlaps with the ultrasound image 30B acquired after target therapy.

Referring again to FIG. 4, the storage unit 280 may store data or algorithms needed for operations of the ultrasonic imaging apparatus 20. For example, the storage unit 280 may store a target image that is subject to image registration, the ultrasound image 30A and the registered image 40A acquired before target therapy, and the ultrasound image 30B and the registered image 40B acquired after target therapy. Also, the storage unit 280 may store a look-up table that represents a mapping relationship between brightness of ultrasound contrast agents which are displayed in the ultrasound image 30A and an actual amount of ultrasound contrast agents.

The storage unit 280 may be Read Only Memory (ROM), Random Access Memory (RAM), Programmable Read Only Memory (PROM), Erasable Programmable Read Only Memory (EPROM), a flash memory, a hard disk drive, an optical disk drive, or a combination of two or more of the above-mentioned devices. However, the storage unit 280 is not limited to these, and may be any other storage device well-known in the art.

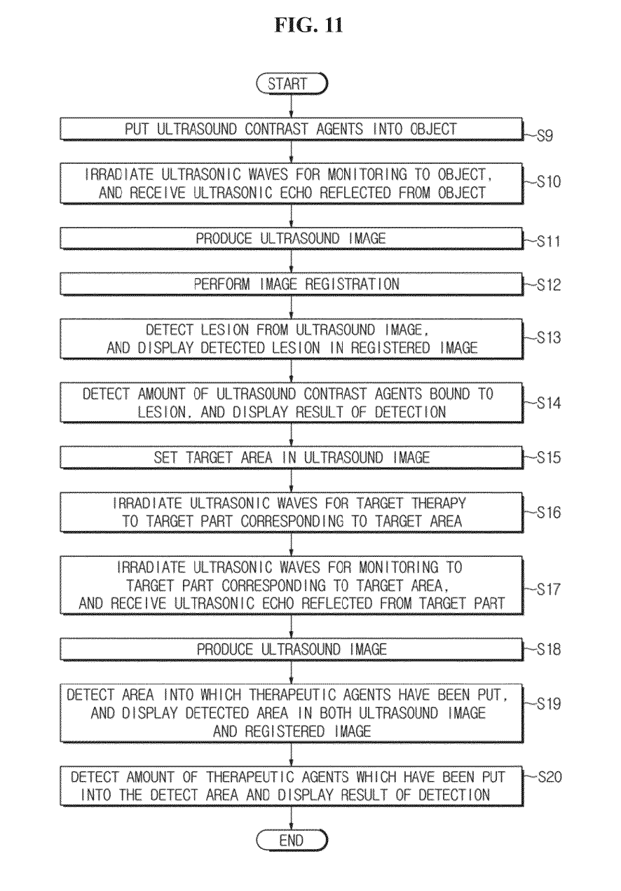

FIG. 11 is a flowchart illustrating a control method of the ultrasonic imaging apparatus 20, according to an exemplary embodiment. The following description will be given with reference to FIGS. 4, 7, 8, 9, and 10.

First, ultrasound contrast agents may be put into the object 10 at operation S9.

Then, ultrasonic waves for monitoring may be irradiated to the object 10, and ultrasonic echoes reflected from the object 10 may be received at operation S10. The operation of irradiating ultrasonic waves and receiving ultrasonic echoes may be performed by one or more ultrasonic elements T, for example, one or more ultrasonic transducers. The ultrasonic elements T may convert the received ultrasonic echoes into electrical signals, and output reception signals. The reception signals output from the ultrasonic elements T may be amplified and filtered, and then converted into digital signals. The reception signals converted into the digital signals may be received and focused by the receive beamformer 260.

Thereafter, an ultrasound image may be produced based on the reception signals focused by the receive beamformer 260 at operation S11. The ultrasound image may be produced by the image producer 271 of the image processor 270.

Thereafter, image registration may be performed using the ultrasound image as a source image at operation S12. A target image that is registered with the source image may be an ultrasound or non-ultrasound image acquired at a different time or at a different angle. The non-ultrasound image may be a CT image, an MRI image, or a PET image. The image registration may be performed by the image registration unit 272 of the image processor 270.

For example, the image registration unit 272 may detect at least one pattern from each of a source image and a target image, and register the target image with the source image based on patterns having a highest similarity among the patterns detected from the source image and the target image. Referring to FIG. 8, a pattern 31 of the ultrasound image 30A is similar to a pattern 41 of the registered image 40A, and accordingly, image registration is performed based on the patterns 31 and 41.

Meanwhile, the source image, and the registered image which is the target image registered with the source image may be displayed by various methods. For example, the source image and the registered image may be displayed side by side in a display area, as shown in FIG. 8. As another example, the source image and the registered image may be displayed to overlap each other, after at least one attribute selected from at least one attribute of the source image and at least one attribute of the registered image is adjusted. Attributes of an image may include transparency and color.

If the image registration is completed, a lesion 32 may be detected from the ultrasound image 30A, and the detected lesion 32 may be displayed to overlap with a lesion 42 of the registered image 40A at operation S13. Operation S13 may be performed by the detector 273 of the image processor 270. A lesion to which ultrasound contrast agents have been bound is shown brightly in the ultrasound image 30A. Accordingly, the detector 273 may detect an area including at least one pixel having a brightness value greater than a reference value, or an area including at least one pixel having a brightness value belonging to a reference range, in the ultrasound image 30A, and determine the detected area as a lesion.

Thereafter, ultrasonic attenuation of the ultrasound image 30A may be compensated for, an amount of ultrasound contrast agents bound to the lesion may be detected, and the result of the detection may be displayed through the information display window 36 at operation S14. Operation S14 may be performed by the detector 273 of the image processor 270.

As described above, since ultrasonic waves attenuation is more significant at a location deeper from the skin of the body or at a location more distant in the lateral direction, a lesion located deeper from the body of the skin is shown darker in the ultrasound image 30A, and a lesion located more distant in the lateral direction is also shown darker in the ultrasound image 30A. Accordingly, the detector 273 may compensate for ultrasonic attenuation of the ultrasound image 30A according to the axial-directional and lateral-directional locations of the lesion.

Then, an amount of ultrasound contrast agents bound to the lesion may be detected with reference to the brightness of the lesion and the look-up table. The more an amount of ultrasound contrast agents bound to the lesion is, the brighter the lesion is shown in the ultrasound image 30A. Accordingly, the detector 273 may detect the brightness of the lesion detected from the ultrasound image 30A, and then search for an amount of ultrasound contrast agents corresponding to the detected brightness in the look-up table. Information about the found amount of ultrasound contrast agents may be displayed through the information display window 36.

Thereafter, if a target area 34 is set in the ultrasound image 30A at operation S15, ultrasonic waves for target therapy may be irradiated to a target part corresponding to the target area 34 at operation S16. The ultrasonic waves for target therapy, irradiated to the target part, may make a part of bubbles of the ultrasound contrast agents burst. As a result, therapeutic agents may be delivered to tissue of the target part. In order to make the bubbles of the ultrasound contrast agents burst, ultrasonic waves of a predetermined resonance frequency, or ultrasonic waves of a predetermined intensity or more may be irradiated to the target part.

In operation S16, the controller 240 may control the transmit beamformer 250 such that ultrasonic waves generated from one or more ultrasonic elements T can be focused to the target part.

For example, the controller 240 may control the transmit beamformer 250 such that when the target area 34 is set in the ultrasound image 30A, ultrasonic waves can be focused to a target part corresponding to the target area 34.

As another example, the controller 240 may control the transmit beamformer 250 such that ultrasonic waves can be focused to a target part corresponding to the target area 34 after a predetermined time period has elapsed from when the target area 34 has been set in the ultrasound image 30A.

As still another example, the controller 240 may control the transmit beamformer 250 such that ultrasonic waves can be focused to a target part corresponding to the target area 34 when a predetermined instruction or command is received through the input unit 210 after the target area 34 is set in the ultrasound image 30A.

As described above, the ultrasonic waves may be irradiated by one or more ultrasonic elements T. The ultrasonic elements T may convert received ultrasonic echoes into electrical signals, and output reception signals. The reception signals output from the ultrasonic elements T may be amplified and filtered, and then converted into digital signals. The reception signals converted into the digital signals may be received and focused by the receive beamformer 260.

Thereafter, ultrasonic waves for monitoring, not aimed at making bubbles burst, may be irradiated to the target part corresponding to the target area 34, and ultrasonic echoes reflected from the target part may be received at operation S17.

If the reception signals focused by the receive beamformer 260 are output, an ultrasound image 30B may be produced based on the reception signals at operation S18. Operation S18 may be performed by the image producer 271 of the image processor 270. The ultrasound image 30B produced by the image producer 271 may be displayed in the display area.

Thereafter, the ultrasound image 30A acquired before target therapy may be compared to the ultrasound image 30B acquired after target therapy to detect an area 35 into which therapeutic agents have been put, and the detected area 35 may be displayed in both the ultrasound image 30B and the registered image 40B at operation S19. Operation S19 may be performed by the detector 273 of the image processor 270. If ultrasonic waves are irradiated to be focused to the target part, the ultrasound contrast agent particles burst by the irradiated ultrasonic waves, so that a part in which the ultrasound contrast agent particles have burst is shown dark in the ultrasound image 30B. Accordingly, the detector 273 may detect an area with reduced brightness in the ultrasound image 30B acquired after target therapy, and determine the detected area as an area 35 into which therapeutic agents have been put.

Thereafter, the detector 273 may detect an amount of the therapeutic agents which have been put into the detected area 35, and display information about the detected amount of the therapeutic agents through the information display window 36 at operation S20. The amount of the therapeutic agents which have been put into the detected area 35 may be indicated by how much darker the detected area 35 is shown as compared to the corresponding area in the ultrasound image 30A acquired before target therapy.

The control method of the ultrasonic imaging apparatus 20 has been described with reference to FIG. 11. In FIG. 11, operations S9 to S14 are first monitoring operations for monitoring a lesion to which ultrasound contrast agents have been bound in an ultrasound image, operations S15 and S16 are target therapy operations for making bubbles of ultrasound contrast agents at a target part burst, and operations S17 to S20 are second monitoring operations for monitoring the results of the target therapy.

Although not illustrated in FIG. 11, after operation S20, the target therapy operations S15 and S16 and the second monitoring operations S17 to S20 may be repeatedly performed. For example, the target therapy operations S15 and S16 and the second monitoring operations S17 to S20 may be repeatedly performed until all ultrasound contrast agent bubbles at the target part burst.

Although a few exemplary embodiments have been shown and described, it would be appreciated by those skilled in the art that changes may be made in these exemplary embodiments without departing from the principles and spirit of the exemplary embodiments, the scope of which is defined in the claims and their equivalents.

What is claimed is:

1. An ultrasonic imaging apparatus comprising:
an inputter configured to receive a command for setting, in a first ultrasound image, a target area of an object to which target therapy is to be applied, the first ultrasound image showing a lesion to which ultrasound contrast agents comprising therapeutic agents have been bound within the object; and
an image processor configured to compare the first ultrasound image to a second ultrasound image acquired after ultrasonic waves have been irradiated to a target part corresponding to the target area, and to detect at least one of an area into which the therapeutic agents have been delivered and an amount of the therapeutic agents delivered into the area.

2. The ultrasonic imaging apparatus according to claim 1, wherein the image processor further comprises a detector configured to compensate for ultrasonic attenuation of the first ultrasound image and the second ultrasound image according to a location of the lesion, and to detect an area with reduced brightness in the second ultrasound image compared to a corresponding area in the first ultrasound area, as the area into which the therapeutic agents have been delivered.

3. The ultrasonic imaging apparatus according to claim 2, wherein the detector is configured to display an area dividing line indicating the detected area in the second ultrasound image in order to distinguish the detected area from a remaining area other than the detected area of the second ultrasound image.

4. The ultrasonic imaging apparatus according to claim 2, wherein the detector is configured to detect the amount of the therapeutic agents delivered into the detected area, based on a reduced degree in brightness of the detected area.

5. The ultrasonic imaging apparatus according to claim 4, further comprising a display configured to display information about the amount of the therapeutic agents delivered into the area.

6. The ultrasonic imaging apparatus according to claim 1, wherein the image processor further comprises a detector configured to compensate for ultrasonic attenuation of the first ultrasound image according to a location of the lesion, and to detect an area including at least one pixel having a brightness value greater than a reference value in the first ultrasound image, as the lesion.

7. The ultrasonic imaging apparatus according to claim 6, wherein the detector is configured to detect an amount of the ultrasound contrast agents bound to the detected lesion, based on brightness of the detected lesion.

8. The ultrasonic imaging apparatus according to claim 7, further comprising a display configured to display information about the amount of the ultrasound contrast agents bound to the detected lesion.

9. An ultrasonic imaging apparatus comprising:
a display configured to display a first ultrasound image showing a lesion to which ultrasound contrast agents comprising therapeutic agents have been bound within an object, and a registered image obtained by registering a predetermined target image with the first ultrasound image;
an inputter configured to receive a command for setting, in the first ultrasound image, a target area to which target therapy is to be applied to the object; and
an image processor configured to compare the first ultrasound image to a second ultrasound image acquired after ultrasonic waves have been irradiated to a target part corresponding to the target area, and to detect at least one of an area into which the therapeutic agents of the ultrasound contrast agents have been delivered, and an amount of the therapeutic agents delivered into the area.

10. The ultrasonic imaging apparatus according to claim 9, wherein the image processor further comprises a detector configured to compensate for ultrasonic attenuation of the first ultrasound image and the second ultrasound image according to a location of the lesion, and to detect an area with reduced brightness in the second ultrasound image compared to a corresponding area in the first ultrasound area, as the area into which the therapeutic agents have been delivered.

11. The ultrasonic imaging apparatus according to claim 10, wherein the display is configured to display an area dividing line indicating the detected area in the second ultrasound image in order to distinguish the detected area from a remaining area of the second ultrasound image other than the detected area, and display an area dividing line indicating an area of the registered image, corresponding to the detected area, in the registered image.

12. The ultrasonic imaging apparatus according to claim 10, wherein the detector is configured to detect the amount of the therapeutic agents delivered into the detected area, based on a reduced degree in brightness of the detected area.

13. The ultrasonic imaging apparatus according to claim 12, wherein the display is configured to display information about the amount of the therapeutic agents delivered into the detected area.

14. The ultrasonic imaging apparatus according to claim 9, wherein the image processor further comprises a detector configured to compensate for ultrasonic attenuation of the first ultrasound image according to a location of the lesion, and to detect an area including at least one pixel having a brightness value greater than a reference value in the first ultrasound image, as the lesion.

15. The ultrasonic imaging apparatus according to claim 14, wherein the display is configured to display the detected lesion as overlapping with the registered image at a location corresponding to the detected lesion in the registered image.

16. The ultrasonic imaging apparatus according to claim 14, wherein the detector is configured to detect the amount of the ultrasound contrast agents bound to the lesion, based on brightness of the detected lesion.

17. The ultrasonic imaging apparatus according to claim 15, wherein the display is configured to display information about the amount of the ultrasound contrast agents bound to the lesion.

18. The ultrasonic imaging apparatus according to claim 9, wherein the display is configured to display, in at least one area among the target area and an area of the registered image corresponding to the target area, an area dividing line for distinguishing the at least one area from a remaining area other than the at least one area.

19. The ultrasonic imaging apparatus according to claim 9, wherein the display is configured to display, in at least one area among an area into which the therapeutic agents have been delivered and an area of the registered area corresponding to the area into which the therapeutic agents have been delivered, an area dividing line for distinguishing the at least one area from a remaining area other than the at least one area.

20. The ultrasonic imaging apparatus according to claim 9, wherein the display is configured to display the first ultrasound image and the registered image as overlapping with each other, after at least one attribute selected from at least one attribute of the first ultrasound image and at least one attribute of the registered image is adjusted.
